(19) United States
(12) Patent Application Publication
(10) Pub. No.: US 2015/0094593 A1
(43) Pub. Date: Apr. 2, 2015

(54) Cuff for Accentuating Venous Flow

(71) Applicants: Lindsay S. Machan, Vancouver (CA); Daniel Gelbart, Vancouver (CA)

(72) Inventors: Lindsay S. Machan, Vancouver (CA); Daniel Gelbart, Vancouver (CA)

(21) Appl. No.: 14/040,990

(22) Filed: Sep. 30, 2013

(51) Int. Cl. A61B 8/00; A61B 8/06

(52) U.S. Cl. CPC A61B 8/403; A61B 8/06
USPC 600/454; 600/437

(57) ABSTRACT

A foot operated air pump rapidly inflates a pneumatic cuff wrapped around the leg of a patient. At the moment of inflation a pressure switch senses the increase in air pressure and sends a timing signal to an ultrasonic imaging system.

### FIELD OF INVENTION

The invention is in the medical field, and in particular in the field of using ultrasound for evaluating vascular conditions such as DVT.

### BACKGROUND OF THE INVENTION

In certain medical procedures it is desired to momentary squeeze the blood from one part of the patient's body and observe its flow. An example is the use of ultrasound to assess Deep Vein Thrombosis (DVT). Traditionally this is performed by a doctor or technician using one hand to squeeze the veins in the calf of the patient and using the other hand to position the ultrasound probe. This procedure is difficult to do as both probe positioning and squeezing have to be done at the same time. Furthermore, the ultrasonic scan has to be synchronized with the squeezing, requiring three events to take place at the same time. The physician or technician performing the procedure must stretch to restrict blood flow through the vein at the same time, which is very and can cause back strain in some people. It is an objective of this invention to greatly simplify this procedure and improve the timing accuracy of starting the ultrasound scan as well as make the system more ergonomic. A further objective is to provide a simple low cost system for the momentary squeezing of a body part.

### Prior Art

There exist devices to automatically detect DVT using pneumatic cuffs; however these methods use plethysmography (Maskell, Tumey U.S. Pat. No. 5,991,654, Amtex Venometer). As the name suggests, strain gauge plethysmography uses a strain gauge (often made of mercury) to detect the circumference of the patient's calf while blood flow is restricted by a pressure cuff. This differs from our disclosed invention, as we are not using plethysmography to detect DVT. Wang et al. recommend using ultrasound for diagnosing DVT before the more invasive gold standard procedure, venography, is attempted and Heijboer et al. conclude that contrast venography cannot be performed in approximately 20% of patients and that compression ultrasonography is a valid alternative. Impedance plethysmography is another non-invasive medical test for DVT that measures small impedances changes. Impedance plethysmography measures electrical changes on the limbs of the patient, which reflects blood flow changes, but in a comparison done by Wells et al., ultrasonography was found to have significantly fewer false negatives and was comparable to radiographic venography.

Friedman et al. (U.S. Pat. Nos. 8,016,761 and 8,043,223) have previously disclosed an invention that automatically uses pressure cuffs and ultrasound to measure endothelial dysfunction. Friedman et al.'s system requires a blood pressure sensor and a pulse oximeter. Our invention differs as we propose a system that requires a pressure sensor to determine when the cuff is sufficiently inflated or similar method to automatically deflate the cuff.

Current methods of using Doppler ultrasound imaging for diagnosing DVT requires the technician to use their hand to obstruct blood, which is cumbersome and can result in injury after repeated use. Furthermore, the popular non-invasive method called impedance plethysmography has been shown to be ineffective in comparison to ultrasonic methods (Wells). Venography, the current gold standard, is invasive, time consuming and requires exposure to ionizing radiation. Our invention is not only safe and easy to use, but it allows the procedure to be done with a simple ergonomic device that prevents repetitive use and strain injuries.

McEwen (U.S. Pat. No. 8,425,426) discloses an invention that uses a pressurized tourniquet that automatically monitors the limb occlusion pressure. Tourniquets, however are designed to operate and occlude blood flow for an extended amount of time, whereas our device has a means of ensuring the blood is only momentarily occluded.

We disclose an invention that uses ultrasound imaging in conjunction with a synchronized pneumatic cuff with a release valve to automatically capture Doppler ultrasound images that can be used to diagnose DVT.

### SUMMARY OF THE INVENTION

A foot operated air pump rapidly inflates a pneumatic cuff wrapped around the leg of a patient. At the moment of inflation a pressure switch senses the increase in air pressure and sends a timing signal to an ultrasonic imaging system.

### BRIEF DESCRIPTION OF THE DRAWINGS

FIG. 1 shows a general view of the system without ultrasound synchronization.

FIG. 2 shows a cross section of a simple pump that can be used in the invention.

FIG. 3 shows a general view of the system.

### DETAILED DESCRIPTION

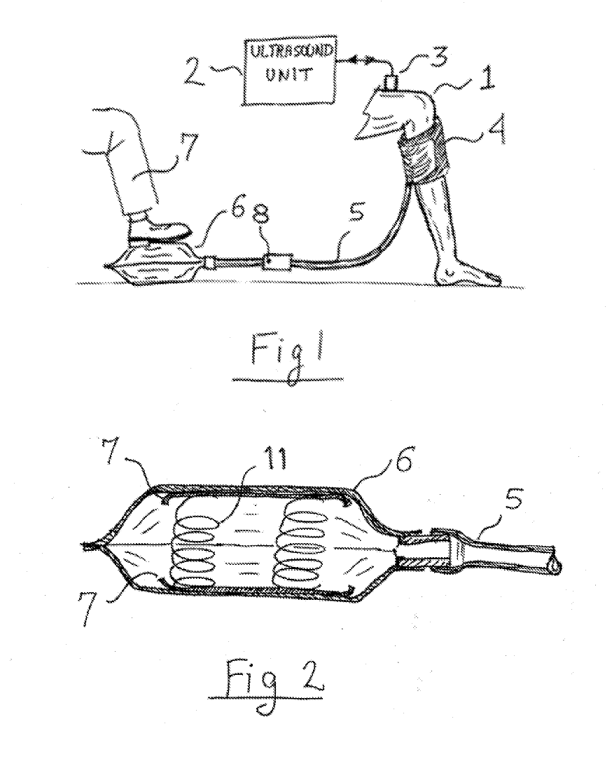

The disclosed invention is a system for rapidly squeezing a body part, such as a leg, and is shown in FIG. 1, to aid in the of diagnosis of vascular disorders. In order to monitor the condition of a patient's leg 1, the calf is momentarily squeezed by a pneumatic cuff 4 and the blood flow is monitored by an ultrasound machine 2 using a transducer 3. Similar ultrasonic procedures are well known in the art, for example for assessing DVT. Cuff 4 is connected to an air pump 6 via hose 5. Air pump 6 can be as simple as a rubber bag, bellows or piston pump. It can also be any type of motorized air pump. Hose 5 can also be connected to an external air supply. In such a case pump 6 is simply replaced by an air valve that can be electrically or mechanically operated. The advantage of using an external air supply is that the pressure generated by the cuff is always the same. When the user steps with foot 7 on air pump 6, cuff 4 inflates. When foot 7 is removed, cuff 4 deflates. Before the physician or technician inflates the cuff 4, they will position an ultrasound probe and start acquiring images. Once in position, the user will inflate the cuff 4 and remove their foot 7, allowing the cuff to deflate. A benefit of this system is that we do not need to use a blood pressure sensor, which allows us to perform procedures on patients that might otherwise not be candidates. When using a simple bag type pump as shown in FIG. 1 it is desired to include a small bleed hole 8. This hole serves a dual purpose: it prevents cutting off blood flow by squeezing the leg for prolonged periods (pressure will drop as air leaks out of hole 8), and it allows the bag to fill with air in case it was stored in the flattened position. In order to keep bag pump 6 full of air when not in use, an internal spring keeps it in the fully inflated state. This is shown in FIG. 2. Springs 11 keep metal plates 7 apart and hold the bag fully inflated until stepped on.

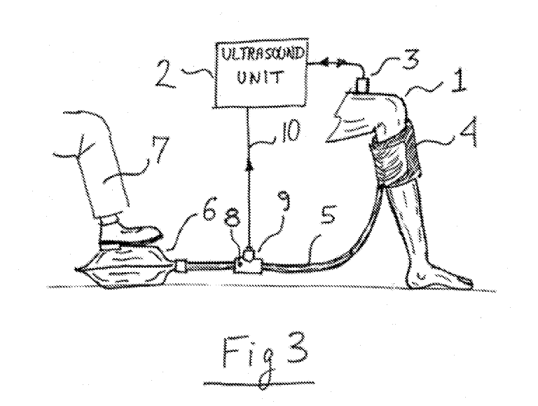

FIG. 3 is a similar configuration that shows an air pressure switch 9 that is activated at a pre-set pressure and sends a synchronization signal to ultrasound unit 2 via wire 10. A variable delay inside unit 2 allows changing the relative timing between squeezing the leg and starting the ultrasound scan. This configuration of the invention simplifies the user interface, as the ultrasound frames containing the pertinent diagnostic information is automatically captured and shown on the display, reducing the overall information displayed to the user. The preferred embodiment of this invention synchronizes an inflatable cuff with the acquisition of ultrasonic images, however, it is foreseeable that another device that restricts blood flow, such as a non-inflatable tourniquet could be used to momentarily occlude the blood flow. It is also foreseeable that the device can be used without automatic synchronization of the cuff with the blood flow monitoring ultrasound equipment.

Cuff 4 is similar to the standard cuff used to measure blood pressure, with the exception of the additional air release mechanism 8. It is wrapped around the leg and held in place by a pressure sensitive fastener such as hook and loop. The unit was tested with such a cuff (removed from a blood pressure gauge) and a standard rubber hot water bottle as a pump with excellent results. The bleed hole 8 was about 0.5 mm in diameter. Hose 5 needs to be of substantial diameter, to allow rapid inflation and deflation. In the test the hose was 1.5 meters long with an inside diameter of 12 mm. Pressure switch 9 was set to 0.01 atmospheres.

Clearly gases or fluids other than air can be used to communicate the pressure from the doctor's foot to the patient's leg. For example, water can be used instead of air. In such a case bleed hole 8 is not used. Also, a direct mechanical system can be used to transmit the pressure from the doctor's leg to the patient. By the way of example, a cable operated system similar to the cables operating the brakes on a bicycle can be used. Pressing on a pedal can create tension a cable. The other end of this cable can be wrapped around the patient's calf. Increasing tension in the cable will squeeze the patient's calf. Similarly, the cuff and pump system can be actuated by a means other than the user's foot or leg. While the preferred embodiment is a pneumatic system, the invention covers all devices for momentary squeezing a patient's limb when activated by a user in order to assist diagnostic procedures using ultrasound. Furthermore, we describe an air pressure switch to aid in synchronizing the ultrasound device, however, the invention covers all methods of synchronizing the pneumatic cuff and imaging device, whether automatic or otherwise.

One advantage of using an external air supply and an air valve instead of a pump is that the system no longer needs a large force to activate the cuff. This allows to use switches other than foot operated ones. For example, a push button can be located on, or next to, the ultrasonic transducer, allowing the user to position the transducer and activate the cuff by pressing a button, or even pressing down the transducer.

The disclosed invention is intended to be used in the diagnosis of DVT, however, it is feasible that it can be used for diagnosing other vascular disorders. It should also be understood that, although we use the terms pneumatic and air, the system can be made with an equally suitable substance.

It should also be noted that we use the term leg of the patient, but it is feasible that certain disorders and diagnostic tests may use the cuff on other parts of the patient's body, such as the arm.

We claim:

1. A device for diagnosing vascular disorders that comprises a flexible member wrapped around a patient's limb and a means of temporarily pressurizing said flexible member while blood flow is ultrasonically monitored.

2. A device as in claim 1 that pressurizes said flexible member with a fluid.

3. A device as in claim 1 that automatically releases the pressure of said flexible member.

4. A device as in claim 1 that automatically captures a diagnostically relevant image or images from an ultrasound machine and either displays said information, stores said information or both.

5. A device as in claim 1 that automatically captures a salient ultrasound image or images and analyzes said images for symptoms of vascular disorders.

6. A method of diagnosing vascular disorders that uses a flexible member wrapped around a patient's limb and a means of temporarily pressurizing said flexible member while blood flow is ultrasonically monitored.

7. A method as in claim 6 wherein the flexible member is pressurized with a fluid.

8. A method as in claim 6 wherein said pressure is automatically released.

9. A method as in claim 6 that automatically captures a diagnostically relevant image or images from an ultrasound machine and either displays said information, stores said information or both.

10. A method as in claim 6 that automatically captures a salient ultrasound images and analyzes said images for symptoms of vascular disorders.

11. A device for diagnosing vascular disorders that comprises a flexible member wrapped around a patient's limb, a means of temporarily pressurizing said flexible member and a synchronized ultrasound acquisition device that is used to monitor blood flow.

12. A device as in claim 11 that pressurizes said flexible member with a fluid.

13. A device as in claim 11 that automatically releases the pressure of said flexible member.

14. A device as in claim 11 that automatically syncronizes the pressurization of said flexible member and the ultrasound acquisition device.

15. A device as in claim 11 that uses Doppler-based ultrasound to monitor blood flow in a patient.

16. A device as in claim 11 that automatically captures a diagnostically relevant image or images from an ultrasound machine and either displays said information or stores it.

17. A device as in claim 11 that automatically captures one or more salient ultrasound images and analyzes said images for symptoms of vascular disorders.
